(19) United States
(12) Patent Application Publication
(10) Pub. No.: US 2017/0224202 A1
(43) Pub. Date: Aug. 10, 2017

(54) ROTATABLE STABILIZER FOR USE OF CATHETER INSIDE AN ENDOSCOPE

(71) Applicant: KONINKLIJKE PHILIPS N.V., EINDHOVEN (NL)

(72) Inventors: HAO SU, HARTSDALE, NY (US); GREGORY COLE, OSSINING, NY (US); VIJAY PARTHASARATHY, LEXINGTON, MA (US)

(21) Appl. No.: 15/518,507

(22) PCT Filed: Oct. 20, 2015
(86) PCT No.: PCT/IB2015/058065

§ 371 Date: Apr. 12, 2017

Related U.S. Application Data
(60) Provisional application No. 62/067,479, filed on Oct. 23, 2014.

(51) Int. Cl. A61B 1/018; A61B 1/267; A61B 90/50; A61M 25/01; A61B 1/00; A61B 17/00

(52) U.S. Cl. CPC A61B 1/018; A61B 1/00045; A61B 1/00009; A61B 1/00128; A61B 17/00234; A61B 90/50; A61M 25/0113; A61B 1/2676; A61B 2017/00199; A61B 2017/00221; A61B 2017/0034; A61B 2017/00477; A61B 2017/0069; A61B 2017/00991

(57) ABSTRACT

An instrument positioning mechanism includes a mounting position (125) on a medical device having a channel configured to permit an instrument (104) to pass therethrough. An instrument support (146) is configured to support a length of the instrument. A pivotal connection (150) is configured to rotatably adjust an angle between the support and the mounting position.

### BACKGROUND

Technical Field

This disclosure relates to medical instruments and more particularly to a medical device that provides improvements for catheter delivery through a channel of an endoscope or similar device.

Description of the Related Art

Catheter-assisted endoscopic interventions can significantly advance the navigation capability of endoscopes. However, endoscope manipulation can be cumbersome and requires multiple operators. This is more evident for catheter-assisted endoscope interventions where an increased number of instruments is needed. In one scenario, a doctor needs to operate the endoscope while a separate operator employs a catheter and potentially an interventional tool. The cumbersome nature of endoscope use can also lead to fatigue of the operators.

In one example, for catheter extended bronchoscopic navigation, a steerable catheter is extended beyond the reach of the bronchoscope working channel when the bronchoscope's diameter is too large to advance inside an airway. In this case, a large portion (30-60 cm long) of the catheter would be left outside of a proximal portion of the working channel where the catheter is inserted. To control the bronchoscope and the steerable catheter is typically a two operator procedure. Bronchoscope navigation control needs three degrees of freedom (DOF): bronchoscope insertion, rotation and flexing. The catheter navigation needs control in at least 2 DOFs: catheter insertion and rotation. To accurately navigate the catheter inside a tortuous lung airway, bimanual manipulation of the two instruments is needed since it is very difficult to perform the required tasks for a single operator. Before the full insertion of the catheter inside bronchoscope, the catheter will often droop down at its proximal end, which makes the catheter manipulation difficult. Moreover, the catheter will buckle if more than 5-6 cm length is not supported making manipulation from a catheter handle difficult during catheter insertion.

A telescopic stabilizer (TS) mechanism consists of several concentric hollow tubes to support the catheter. However, the TS mechanism is fixed at an angle of 40-50 degrees from a centerline of the bronchoscope handle. This causes the telescopic arm to sweep through an inconveniently large area making navigation of the bronchoscope before insertion of the catheter very awkward.

### SUMMARY

In accordance with the present principles, an instrument positioning mechanism includes a mounting position on a medical device having a channel configured to permit an instrument to pass therethrough. An instrument support is configured to support a length of the instrument. A pivotal connection is configured to rotatably adjust an angle between the support and the mounting position.

Another instrument positioning mechanism includes an endoscope having a working channel configured to permit an instrument to pass therethrough. A telescopic stabilizer is configured to support a length of the instrument. The telescopic stabilizer is extendable to adjustably support a length of the instrument. A pivotal connection is configured to rotatably adjust an angle between the telescopic stabilizer and the endoscope. The pivotal connection is hollow or open to receive the instrument.

A method for positioning an instrument includes providing a mounting position on an endoscope having a working channel; mounting an instrument support to the mounting position relative to the working channel by a pivotal connection; passing an instrument into the instrument support, through the pivotal connection and into the working channel; and adjusting an angle of the instrument support relative to the endoscope to provide at least one of reduced space or ease of use for the instrument.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a block/flow diagram showing a system having an assembly for providing a catheter-assisted endoscopic intervention in accordance with one embodiment of the present principles;

FIG. 2A is a diagram showing an instrument support mechanism (telescopic stabilizer) connected to an endoscope with a spherical joint in accordance with one embodiment;

FIG. 2B is an exploded view of the diagram of FIG. 2A in accordance with one embodiment;

FIG. 3A is a diagram showing an instrument support mechanism (telescopic stabilizer) connected to an endoscope with a universal joint in accordance with one embodiment;

FIG. 3B is an exploded view of the diagram of FIG. 3A in accordance with one embodiment;

FIG. 4 is a diagram showing an illustrative exploded magnified view of a universal joint in accordance with one embodiment; and

FIG. 5 is a flow diagram showing a method for positioning an instrument in accordance with illustrative embodiments.

### DETAILED DESCRIPTION OF EMBODIMENTS

In accordance with the present principles, systems and methods are described that overcome the shortcomings of conventional catheter-assisted endoscopic systems. Embodiments in accordance with the present principles permit pivotal mounting of a catheter delivery instrument to an endoscope to permit handheld operation of both the endoscope and the catheter. When inserting a catheter inside an endoscope or bronchoscope for peripheral navigation, a telescopic stabilizer (TS) mechanism may be employed to allow one operator to control two instruments (e.g., the endoscope and the catheter). However, telescopic stabilizer (TS) mechanisms are bulky and interfere with the clinical environment, hampering its intended application.

In accordance with the present principles, a telescopic stabilizer is provided that permits a clinician to navigate a catheter with one hand, while holding and operating an endoscope (e.g., a bronchoscope) with the other hand. This permits the clinician to maintain greater control over the instruments and reduces the need for additional staff during a procedure. The present principles simplify complicated workflows, e.g., a bronchoscopy procedure. Streamlining workflow has the potential to increase the adoption rate of the procedures, reduce the required personage during the procedures and enhance the usability of the telescopic stabilizer to avoid complication in a bustling clinical environment.

The present principles employ a compact and lightweight connection mechanism that can be mounted on or near an endoscope working channel to aid with catheter manipulation. The connection mechanism may include a hollow joint that permits the catheter to pass therethrough. The hollow or open joint connects a telescopic stabilizer to the endoscope and permits the catheter to pass through the telescopic stabilizer and into the working channel.

The present principles may be employed in combination with catheters or other instruments and endoscopes or the like to provide the motion of catheters through a joint connection mounted to the endoscope. The joint connection may include a spherical joint, a universal joint, a revolute joint, etc.

It should be understood that the present invention will be described in terms of catheter-based medical instruments; however, the teachings of the present invention are much broader and are applicable to any flexible, elongated instruments. In some embodiments, the present principles are employed in tracking or analyzing complex biological or mechanical systems. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W), Blu-Ray™ and DVD.

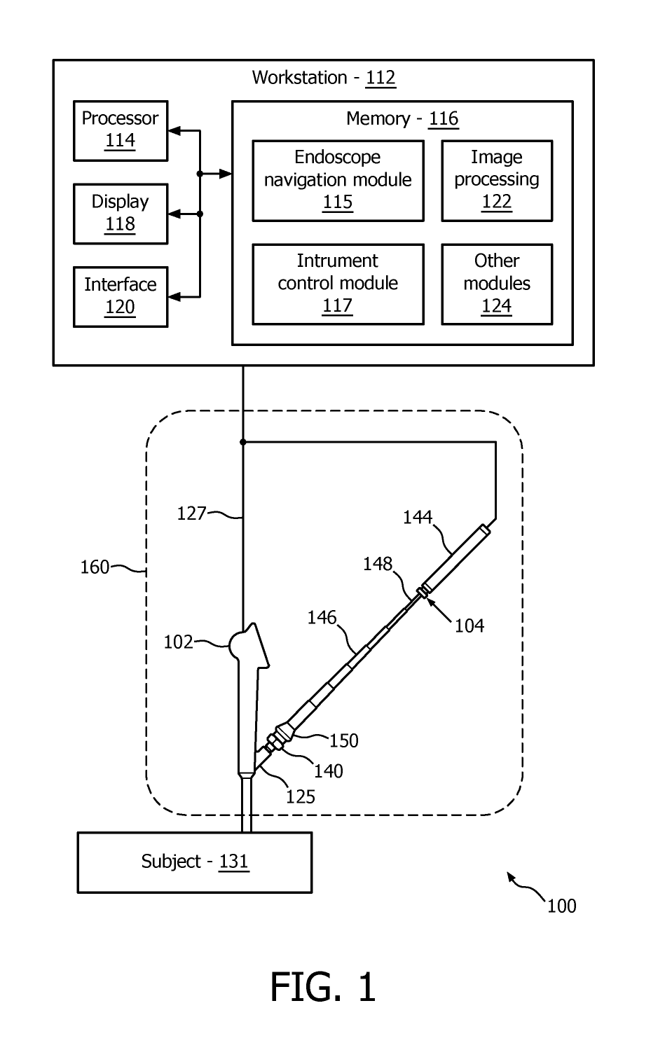

Referring now to the drawings in which like numerals represent the same or similar elements and initially to FIG. 1, a system 100 for performing a procedure, which employs an endoscope mount utilizing a connection joint for instrument control, is illustratively shown in accordance with one embodiment. System 100 may include a workstation or console 112 from which a procedure is supervised, controlled and/or managed. Workstation 112 preferably includes one or more processors 114 and memory 116 for storing programs and applications. Memory 116 may store an endoscope navigation module 115 configured to interpret feedback signals and provide navigation directions for the placement and operation of a mounting device 102, such as an endoscope. The endoscope 102 may be manually controlled, although robotically controlled endoscopes may also be employed. The present principles provide the mounting device 102 with a mounting position 125 for securing another instrument 104.

Memory 116 may also store an instrument control module 117 configured to interpret feedback signals and control the placement and operation of the instrument 104. It should be understood that the endoscope 102 and the instrument 104 may include software and/or hardware (e.g., manual) controls and settings. In addition, although referred to as an endoscope 102 and instrument 104, these devices may include any instruments or devices that are employed in conjunction and should not be construed as limited by the examples given.

Modules 115 and 117 are configured to use the signal feedback (and any other available feedback) to position, reposition or perform other tasks with the endoscope 102 and the instrument 104, respectively. The instrument 104 may include a catheter, a guidewire, a probe, another endoscope, an electrode, a filter device, a balloon device, another medical component, etc.

The endoscope 102 and instrument 104 can communicate with their respective modules 115 and 117 through cabling 127 or wireless communications. The cabling 127 may include fiber optics, electrical connections, other instrumentation, etc., as needed.

In useful embodiments, workstation 112 includes modules to perform different tasks during a procedure. These modules may include an image processing module 122 to process images collected by the endoscope 102 or instrument 104. Other modules 124 may include application specific controls and measurements systems to control power, measure parameters, etc.

Workstation 112 preferably includes a display 118 for viewing internal images of a subject (patient) or volume 131. Display 118 may also permit a user to interact with the workstation 112 and its components and functions, or any other element within the system 100. This is further facilitated by an interface 120 which may include a keyboard, a mouse, a joystick, a haptic device, or any other peripheral or control to permit user feedback from and interaction with the workstation 112.

In a particularly useful embodiment, the instrument 104 includes a catheter that is positioned within a working channel of the endoscope 102. A telescopic stabilizer 146 may be employed to support the catheter 104 inside the telescopic stabilizer 146 as it enters the working channel or working channel adapter 140. The telescopic stabilizer 146 may receive a handle adapter 148 configured to protect the catheter 104 at or near a catheter handle 144. In addition, the use of the telescopic stabilizer 146 avoids the need for an extra person to operate the catheter 104 since catheter length is available in the telescopic stabilizer 146, and the catheter length can be directly fed into the working channel of the endoscope 102 without drooping or buckling. Therefore, one-handed catheter operation is provided.

A mounting position 125 may be coupled to a pivotal connection or joint 150, e.g., a spherical joint, a universal joint, a resolute joint, etc. The pivotal connection 150 may be employed to connect the endoscope 102 and the telescopic stabilizer 146. The pivotal connection or joint 150 may include a locking mechanism (not shown). In one embodiment, the joint friction may provide enough stiffness to sustain a set position.

The catheter 104 preferably runs through the telescopic stabilizer 146 and through the joint 150 into a base or mounting position 125 (e.g., on the endoscope 102, although other base mounts or positions may be employed). The joint 150 may be open or hollow to receive the catheter 104. If the base position includes an endoscope 102, the catheter 104 (or other instrument) may run through a working channel of the endoscope 102. In another embodiment, the catheter 104 may be mounted, using the joint 150, to a port or other base.

The telescopic stabilizer 146 is longitudinally expandable and stores a length of a catheter 104 while providing a direction for advancing (or retracting) the catheter 104. In addition, once the catheter 104 is positioned, the telescopic stabilizer 146 may be rotated out of the way using the joint 150. The joint 150 may be lockable with a rotation knob or other object that presses against the joint mechanism(s), although other mechanisms may also be employed.

A handheld assembly 160 may include the endoscope 102, the instrument 104, joint 150, telescopic stabilizer 146, etc. The assembly 160 provides the rotatable telescopic arm or stabilizer 146 to support catheter insertion and permit a clinician to reposition the telescopic stabilizer 146 for more comfortable operation by employing the joint 150. The joint 150 may include, e.g., one or more of a spherical joint mechanism, a universal joint mechanism and a revolute joint mechanism.

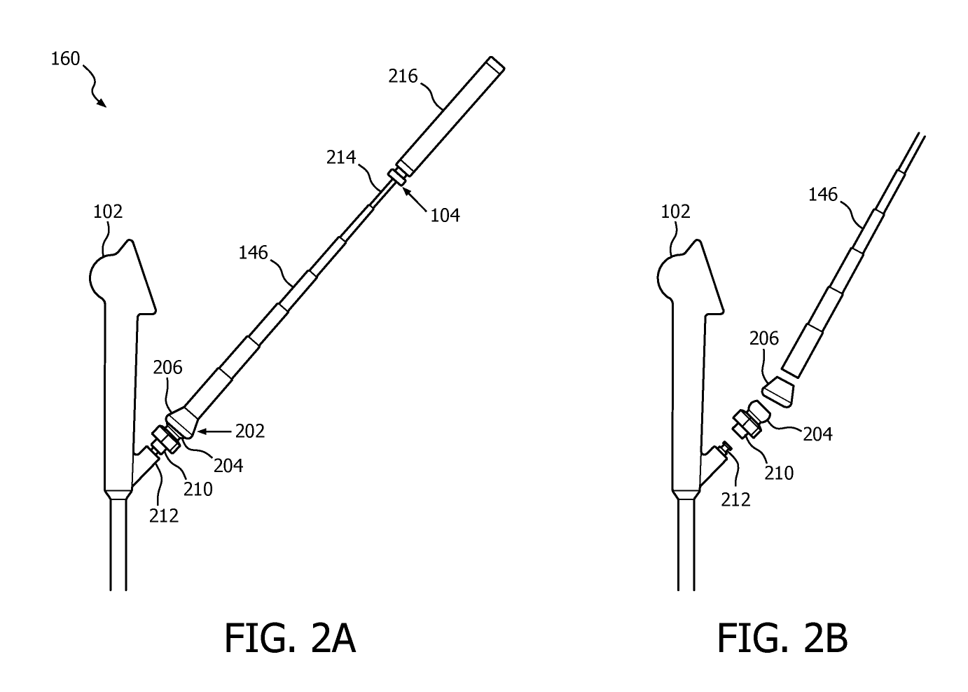

Referring to FIGS. 2A and 2B, an embodiment of a handheld assembly 160 having a rotatable telescopic stabilizer 146 with a spherical joint assembly 202 is illustratively shown. On a distal end of the telescopic stabilizer 146, the spherical joint assembly 202 (including a spherical joint ball 204 and a spherical joint socket 206) connects a working channel 212 and the telescopic stabilizer 146 using a working channel adaptor 210. Since the working channel 212 typically has a female Luer lock feature, the working channel adaptor 210 also has a Luer lock feature to quickly attach or detach the overall telescopic stabilizer 146 to the working channel 212. On a proximal end of the telescopic stabilizer 146, a handle adaptor 214 connects a catheter handle 216 with the stabilizer 146 while allowing catheter rotation. The stabilizer 146 avoids catheter kinking when the catheter handle droops or whenever the catheter shaft is vulnerable to kinking. The catheter handle 216 and telescopic stabilizer 146 can connect as a single piece in the axial direction using the handle adaptor 214. This reduces the degrees of freedom and prevents the catheter 104 from sliding inside the stabilizer 146.

The spherical joint assembly 202 provides three degrees of freedom (DOF) for rotation motion (e.g., roll, pitch and yaw). The spherical joint ball 204 and the spherical joint socket 206 preferably have a tight fit which provides appropriate friction to permit free rotation of the joint assembly 202 when friction is overcome, but also provides a fixed posture when the mechanism is rotated to some fixed position. Since this configuration permits rotation within a plane of the endoscope 102, the telescopic stabilizer 146 and accessories attached thereto could be aligned almost in parallel with a main axis of the endoscope handle (102) to reduce the overall footprint, which is especially useful during endoscope rotation. Also, since 3-DOF rotation is provided, a wide range of directions/angles can be achieved to allow very flexible and dexterous manipulation of the catheter 104.

FIG. 2B shows details of spherical joint assembly 202 including the spherical joint ball 204 and the spherical joint socket 206 with the endoscope working channel 212 and the telescopic stabilizer 146 in an exploded view.

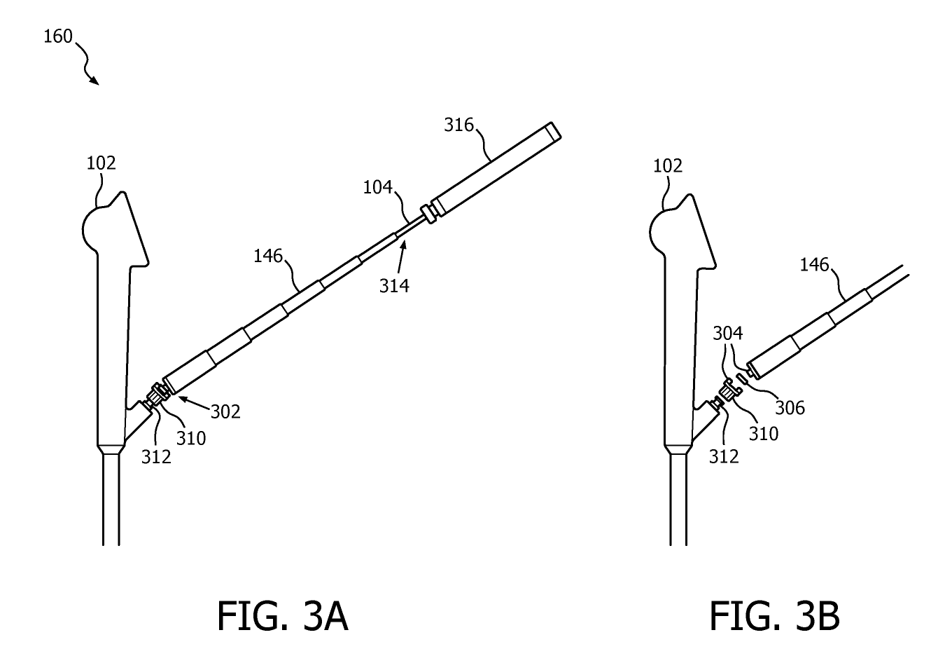

Referring to FIGS. 3A and 3B, an embodiment of a handheld assembly 160 having a rotatable telescopic stabilizer 146 with a universal joint assembly 302 is illustratively shown. In this embodiment, the universal joint assembly 302 is employed to provide a 2-DOF rotation (e.g., pitch and yaw motion). The 2-DOF design may be beneficial to constrain the axial rotation of the catheter 104 since this DOF motion could be controlled by the operator.

On a distal end of the telescopic stabilizer 146, the universal joint assembly 302 (including universal joint bodies 304 and a ring 306, FIG. 3B) connects a working channel 312 and the telescopic stabilizer 146 using a working channel adaptor 310. The working channel adaptor 310 may be configured to be one of the universal joint bodies (304). Since the working channel 312 typically has a female Luer lock feature, the working channel adaptor 310 also has a Luer lock feature to quickly attach or detach the overall telescopic stabilizer 146 to the working channel 312. On a proximal end of the telescopic stabilizer 146, a handle adaptor 314 connects a catheter handle 316 with the stabilizer 146. The stabilizer 146 avoids catheter kinking when the catheter handle droops or whenever the catheter shaft is vulnerable to kinking. The catheter handle 316 and telescopic stabilizer 146 can connect as a single piece in the axial direction using the handle adaptor 314. This reduces the degrees of freedom and prevents the catheter 104 from sliding inside the stabilizer 146.

The universal joint assembly 302 provides two degrees of freedom (DOF) for rotation motion (e.g., pitch and yaw). The universal joint assembly 302 may include a bellows, corrugated tube or other device that permits free movement of the joint assembly 302, but also provides a fixed posture when the mechanism is not being moved by a user to some fixed position. Since this configuration permits rotation within a plane of the endoscope 102, the telescopic stabilizer 146 and accessories attached thereto could be aligned almost in parallel with a main axis of the endoscope handle (102) to reduce the overall footprint, which is especially useful during endoscope rotation. Also, since 2-DOF rotation is provided, a wide range of directions/angles can be achieved to allow very flexible and dexterous manipulation of the catheter 104.

FIG. 3B shows details of universal joint assembly 302 including the universal joint body 304 connected to the stabilizer 146, and the working channel adaptor 310 forming the second universal joint body 304. The ring 306 is disposed within the bodies 304 and depicted in an exploded view.

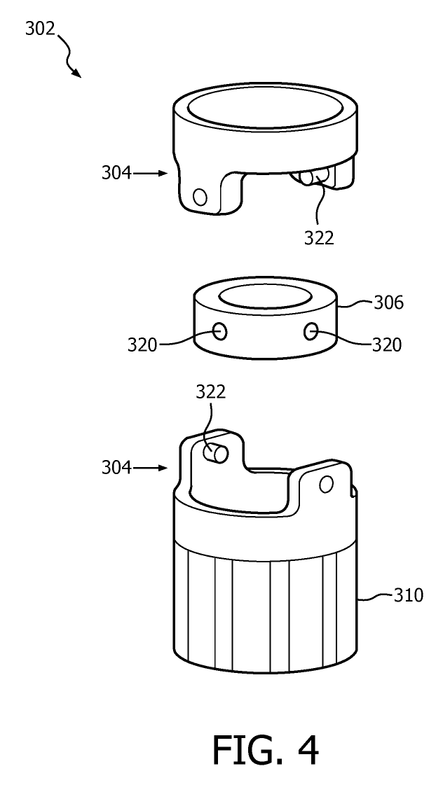

Referring to FIG. 4, an exploded magnified view of a hollow universal joint assembly 302 is illustratively shown. In this design, two universal bodies 304 are connected to a universal joint ring 306. The ring 306 includes four pivot holes 320 to connect with pegs or pins 322 of the bodies 304. Both the ring 306 and the bodies 304 are hollow permitting the catheter (104) to pass through. Note that one of the bodies 304 may also be the working channel adapter 310.

An additional embodiment may include a stripped-down version of the universal joint assembly 302. This embodiment includes a 1-DOF pivot (a revolute joint) instead of a full universal joint. The 1-DOF pivot only allows the telescopic stabilizer 146 to rotate in the plane of the endoscope handle. This design is simple to manufacture and intuitive to operate, while maintaining the benefits described for the other designs. This design could be achieved using only one pair of pins 322 and holes 320 normal to the plane in which pivoting is achieved. This provides a revolute joint, which is in effect one degree of rotational freedom about a revolute axis. Other revolute joint configurations are also contemplated and within the scope of the present principles. For example, hollow tubes or open channels may be pinned together to form a revolute joint between a mounting position and an instrument support.

Applications of the present principles include catheter-based bronchoscope or endoscope procedures that need a supported catheter to assist the procedure or reduce the operational personnel. It should be understood that the present principles may be employed with other instruments in other configurations as well.

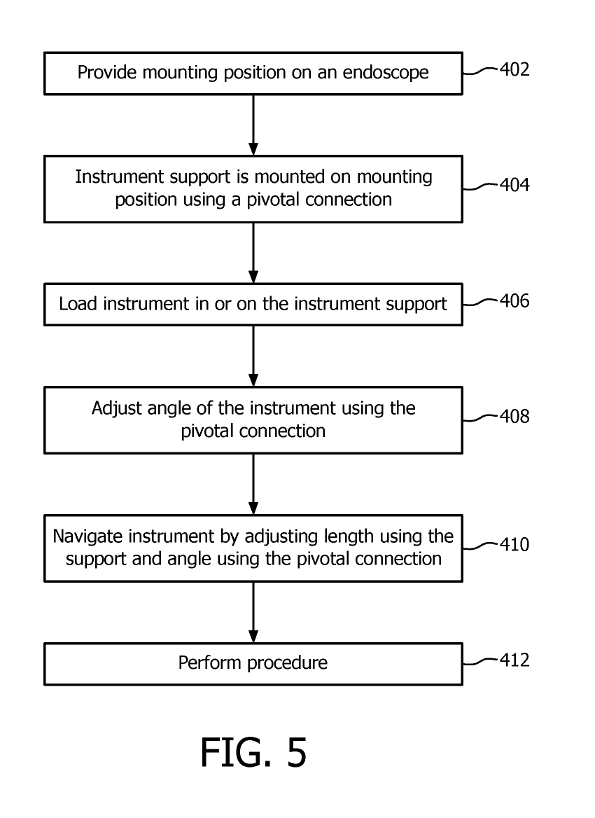

Referring to FIG. 5, a method for positioning an instrument is shown in accordance with illustrative embodiments. In block 402, a mounting position is provided on an endoscope having a working channel. The endoscope may be employed for any number of procedures. In one embodiment, the endoscope includes a bronchoscope for performing a procedure on the lungs. The mounting position may be at or near the working channel and may include a working channel adapter to provide an interface for other components. In block 404, an instrument support is mounted to the mounting position relative to the working channel by a pivotal connection. The pivotal connection is preferably hollow or open to enable an instrument to pass through or over. However, the pivotal connection may not be hollow and the pivotal connection may be placed adjacent to the working channel. The pivotal connection may include at least one of a spherical joint, a universal joint and a revolute joint. Multiple pivotal joints may be employed together and/or in combination to achieve different angles and configurations, as needed.

In block 406, an instrument is loaded (passed into or onto) the instrument support, through or passed the pivotal connection and into the working channel. In block 408, an angle of the instrument support is adjusted relative to the endoscope to provide at least one of reduced space or ease of use for the instrument. By closing the angle of the pivotal connection, the footprint of the overall assembly is reduced. Also, the angle of insertion or retraction of the instrument can be dynamically changed by a user to make repositioning, advancing, retracting, etc. of the instrument easier using the pivotal connection.

In block 410, the instrument is navigated by adjusting a length of instrument support and rotating the instrument support using the pivotal connection. The instrument may include a catheter and the instrument support may include a telescopic stabilizer, a length of the telescopic stabilizer is adjusted to support the catheter. In block 412, a procedure is performed with a single operator. This may include the use of the endoscope concurrently with the use of a rotatable telescopic stabilizer supported catheter or catheters.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for rotatable stabilizer for use of catheter inside an endoscope (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. An instrument positioning mechanism, comprising:
a mounting position on a medical device having a channel configured to permit an instrument to pass therethrough;
an instrument support configured to support a length of the instrument; and
a pivotal connection configured to rotatably adjust an angle between the support and the mounting position and secure a position of the support at an adjusted angle.

2. The mechanism as recited in claim 1, wherein the mounting position is included on an endoscope and the instrument is passed through the endoscope.

3. The mechanism as recited in claim 1, wherein the instrument includes a catheter and the instrument support includes a telescopic stabilizer for supporting the catheter.

4. The mechanism as recited in claim 1, wherein the pivotal connection includes a spherical joint and provides three degrees of freedom for rotation.

5. The mechanism as recited in claim 1, wherein the pivotal connection includes a universal joint and provides two degrees of freedom for rotation.

6. The mechanism as recited in claim 1, wherein the pivotal connection includes a revolute joint and provides one degree of freedom for rotation.

7. The mechanism as recited in claim 1, wherein the instrument support includes a catheter handle adapter on a proximal end portion configured to receive a catheter handle.

8. The mechanism as recited in claim 1, wherein the mounting position includes an endoscope having a working channel and the pivotal connection connects to a working channel adapter.

9. An instrument positioning mechanism, comprising:
an endoscope having a working channel configured to permit an instrument to pass therethrough;
a telescopic stabilizer configured to support a length of the instrument, the telescopic stabilizer being extendable to adjustably support a length of the instrument; and
a pivotal connection configured to rotatably adjust an angle between the telescopic stabilizer and the endoscope and secure a position of the telescopic stabilizer at an adjusted angle, the pivotal connection being hollow or open to receive the instrument.

10. The mechanism as recited in claim 9, wherein the instrument includes a catheter and the telescopic stabilizer supports the catheter to prevent kinking or drooping.

11. The mechanism as recited in claim 9, wherein the pivotal connection includes a spherical joint and provides three degrees of freedom for rotation.

12. The mechanism as recited in claim 9, wherein the pivotal connection includes a universal joint and provides two degrees of freedom for rotation.

13. The mechanism as recited in claim 9, wherein the pivotal connection includes a revolute joint and provides one degree of freedom for rotation.

14. The mechanism as recited in claim 9, wherein the telescopic stabilizer includes a catheter handle adapter on a proximal end portion configured to receive a catheter handle.

15. (canceled)

16. A method for positioning an instrument, comprising:
providing a mounting position on an endoscope having a working channel;
mounting an instrument support to the mounting position relative to the working channel by a pivotal connection;
passing an instrument into the instrument support, through the pivotal connection and into the working channel; and
adjusting an angle of the instrument support relative to the endoscope and securing a position of the instrument support at an adjusted angle to provide at least one of reduced space or ease of use for the instrument.

17. (canceled)

18. (canceled)

19. (canceled)

20. (canceled)
